(19) United States
(12) Patent Application Publication
(10) Pub. No.: US 2006/0194971 A1
(43) Pub. Date: Aug. 31, 2006

(54) Anethole dithiolethione and other dithiolethiones for the treatment of conditions associated with dysfunction of monoamine neruotransmission

(76) Inventors: Benjamin Drukarch, Weesp (NL); Anton N.M. Schoffelmeer, Weesp (NL); Roelof W. Feenstra, Weesp (NL); Marie-Odile Christen, Weesp (FR); Jean-Louis Burgot, Weesp (FR); Marylene Chollet, Weesp (FR); Wouter I. Iwema Bakker, Weesp (NL); Bernard J. Van Vliet, Weesp (NL); Martinus Th.M Tulp, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/357,962

(22) Filed: Feb. 22, 2006

Related U.S. Application Data
(60) Provisional application No. 60/655,428, filed on Feb. 24, 2005.

(51) Int. Cl. A61K 31/496; C07D 409/02; C07D 339/02

(52) U.S. Cl. 544/374; 549/36

(57) ABSTRACT

The present disclosure relates to dithiolethione derivatives as monoamino oxidase inhibitors, in particular MAO-B inhibitors, to methods for the preparation of these compounds and to novel intermediates useful for the synthesis of said dithiolethiones derivatives. The present disclosure also relates to the use of a compound disclosed herein for the manufacture of a medicament giving a beneficial effect. In embodiments of the present disclosure specific compounds disclosed herein are used for the manufacture of a medicament useful in the treatment, amelioration or prevention of conditions associated with dysfunction of monoamine neurotransmission. The compounds have the general formula (1) 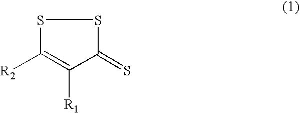 wherein the symbols have the meanings given in the specification.

This application claims benefit of U.S. Provisional Application No. 60/655,428, filed Feb. 24, 2005, the contents of which are incorporated herein by reference. This application also claims the benefit of priority to International Application No. PCT/______, entitled “ANETHOLE DITHIOLETHIONE AND OTHER DITHIOLETHIONES FOR THE TREATMENT OF CONDITIONS ASSOCIATED WITH DYSFUNCTION OF MONOAMINE NEUROTRANSMISSION,” of Benjamin DRUKARCH, Anton N. M. SCHOFFELMEER, Roelof W. FEENSTRA, Marie-Odile CHRISTEN, Jean-Louis BURGOT, Marylene CHOLLET, Wouter I. IWEMA BAKKER, Bernard J. VAN VLIET, and Martinus Th. M. TULP filed ______, which is incorporated herein by reference.

The present disclosure relates to dithiolethiones derivatives as monoamino oxidase inhibitors, including MAO-B inhibitors, to methods for the preparation of these derivative compounds, and to novel intermediates useful for the synthesis of said dithiolethiones derivatives.

The present disclosure also relates to the use of at least one compound disclosed herein for the manufacture of a medicament giving a beneficial effect. A beneficial effect is disclosed herein or apparent to a person skilled in the art from the specification and general knowledge in the art.

The present disclosure also relates to the use of at least one compound of the present disclosure for the manufacture of a medicament for treating or preventing a disease or condition. In addition, the present disclosure relates to at least one new use for the treatment of a disease or condition disclosed herein or apparent to a person skilled in the art from the specification and general knowledge in the art.

In embodiments of the present disclosure, specific compounds disclosed herein are used for the manufacture of a medicament useful in the treatment, amelioration or prevention of conditions associated with dysfunction of monoamine neurotransmission.

Inhibitors of the mitochondrial flavo-enzyme monoamino oxidase (MAO; EC 1.4.3.4) can cause an increase in the levels of norepinephrine, epinephrine, dopamine, tryptamine and serotonin in the brain and other tissues, and thus can cause a wide variety of pharmacological effects mediated by their effects on these neurotransmitters.

Currently available inhibitors such as L-deprenyl, mofegiline, resagiline, lazabemide have a broad range of side-effects including psychiatric (delirium, hallucinations, agitation), cardiovascular (orthostatic hypotension, hypertension) and neurological (sedation, abnormal movements).

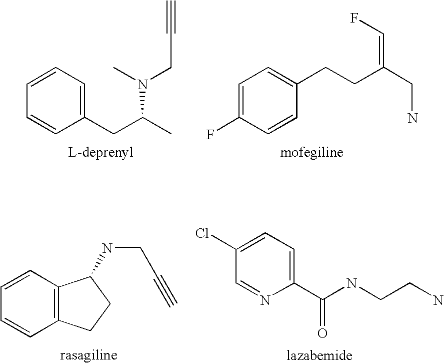

Thus, there is a need to develop new MAO inhibitors, structurally unrelated to those presently available, and which exhibit less side effects.

WO 98/27970 discloses the use of 1,2-dithiol-3-thiones for the treatment of diseases or prevention of cellular damage caused by oxygen-containing radicals. WO 01/09118 discloses dithiolethione compounds for the treatment of neurological disorders and for memory enhancement. These compounds were said to inhibit D-amino acid oxidase (DAAO, E.C. 1.4.3.3), the enzyme that stereoselectively deaminates D-amino acids, thereby generating hydrogen peroxide, a reactive oxygen species. Dithiolethiones have never been shown to inhibit monoamine oxidase, a completely different enzyme.

Surprisingly, it has now been found that dithiolethiones potently inhibit MAO-B activity in cellular extracts derived from cultured rat striatal astroglial cells, whereas no significant effect is observed on MAO-A activity. One aspect of the present disclosure relates to the use of compounds of the general formula (1), 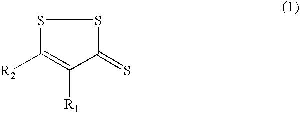
wherein:
R₁ and R₂ are the same or different, and are chosen from hydrogen, alkyl, alkenyl, alkynyl, aryl, fluoro, chloro, bromo, hydroxy, alkyloxy, alkenyloxy, aryloxy, acyloxy, amino, alkylamino, dialkylamino, arylamino, thio, alkylthio, arylthio, cyano, nitro, acyl, amido, alkylamido, and dialkyiamido; or
R₁ and R₂ may together with the carbon atoms to which they are attached form a 5- or 6-membered aromatic or non-aromatic ring containing 0, 1 or 2 heteroatoms chosen from nitrogen, oxygen and sulphur, such as, for example, furan, thiophene, pyrrole, oxazole, thiazole; imidazole, pyrazole, isoxazole, isothiazole, 1,2,3-oxadiazole, 1,2,3-triazole, 1,3,4-thiadiazole, pyridine, pyridazine, pyrimidine or pyrazine rings;
R₁ and R₂ are optionally substituted with at least one substituent chosen from hydrogen, alkyl, alkenyl, alkynyl, aryl, fluoro, chloro, bromo, hydroxyl, alkyloxy, aminoalkyloxy, morpholin-4-yl-alkoxy, piperidin-1-yl-alkyloxy alkenyloxy, aryloxy, acyloxy, amino, alkylamino, dialkylamino, arylamino, thio, alkylthio, arylthio, cyano, oxo, nitro, acyl, amido, alkylamido and dialkylamido,
and tautomers, stereoisomers and N-oxides thereof, as well as pharmacologically acceptable salts, hydrates and solvates of said compounds of formula (1) and its tautomers, stereoisomers and N-oxides, for the preparation of a pharmaceutical composition for the treatment, amelioration or prevention of conditions associated with dysfunction of monoamine neurotransmission.

Embodiments of the present disclosure also relate to the use of compounds of the general formula (1) wherein R₁ and R₂ are the same or different, and are chosen from hydrogen, alkyl or aryl, optionally substituted with at least one atom or group chosen from hydrogen, alkyl, aryl, fluoro, chloro, bromo, hydroxyl, alkyloxy, aryloxy, amino, alkylamino, dialkylamino, thio, oxo and nitro.

At least one embodiment of the present disclosure also relates to the use of 5-(p-methoxyphenyl)-3H-1,2-dithiole-3-thione (anethole ditiolethione, ADT), 3H-1,2-dithiole-3-thione (D3T) and 4-methyl-5-(2-pyrazinyl)-3H-1,2-dithiole-3-thione (oltipraz):

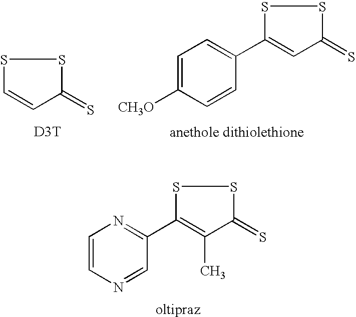

At least one embodiment of the present disclosure uses 5-(p-methoxyphenyl)-3H-1,2-dithiole-3-thione, anethole dithiolethione (ADT), a lipophilic, substituted analogue of 3H-1,2-dithiole-3-thione (D3T), in clinical use for decades as a cholagogue and sialogogue without any major adverse reactions being noted (Christen, M-O., Methods Enzymol., 252, 316-323, 1995).

In another aspect, the present disclosure relates to compounds of formula (1): 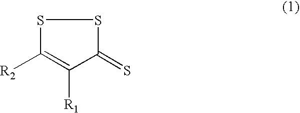
wherein:
R₁ is optionally substituted phenyl and R₂ is chosen from S—CH₂-(4-methyl-phenyl) and subgroups (i), (ii) and (iii): 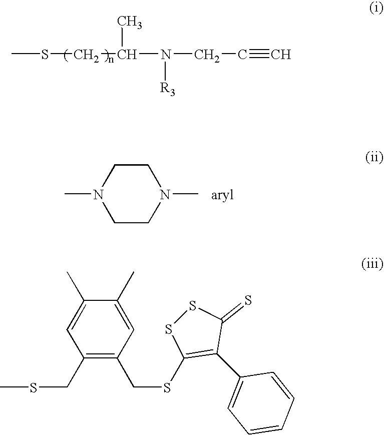
   wherein n is chosen from 2, 3, 4 and 5 and R₃ is chosen from hydrogen and alkyl(C₁₋₃); or
R₁ is 4-hexyloxyphenyl and R₂ is hydrogen; or
R₁ is substituted phenyl and R₂ is chosen from SH and subgroup (iv): 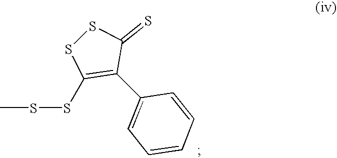
R₁ is hydrogen and R₂ is chosen from —CH═CH-4-(diethylaminophenyl), —CH═CH-(2-quinolyl) and subgroup (v): 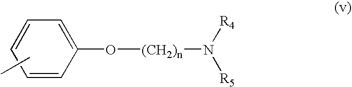
   wherein n is chosen from 2, 3, 4 and 5, and R₄ and R₅ independently are chosen from alkyl(C₁₋₃) groups, or together with the nitrogen atom to which they are attached form a saturated 5- or 6-membered ring, optionally containing a hetero-atom chosen from O, S, and an additional N; or
R₁ is alkyl(C₁₋₃) and R₂ is 1-(2,3-dihydro-1,4-benzodioxin-5-yl)piperazin-4-yl; or
R₁ is cyano and R₂ is —NH—C(O)—NH-phenyl, wherein the phenyl group is optionally substituted; or
R₁ is —SO₂CH₃ and R₂ represents amino;
and tautomers, stereoisomers and N-oxides thereof, as well as pharmacologically acceptable salts, hydrates and solvates of said compounds of formula (1) and its tautomers, stereoisomers and N-oxides.

The present disclosure also relates to racemates, mixtures of diastereomers, as well as the individual stereoisomers of the compounds having formula (1).

In the present disclosure, the following definitions apply to the description of the substituents:

“Alkyl” means C₁₋₃-alkyl.

“Alkenyl” means C₁₋₃-alkenyl.

“Alkynyl” means C₁₋₃-alkynyl.

“Acyl” means alkyl(C₁₋₃)carbonyl, arylcarbonyl or arylalkyl(C₁₋₃)carbonyl.

“Aryl” means furyl, thienyl, pyrrolyl, oxazolyl, thiazolyl, imidazolyl, pyrazolyl, isoxazolyl, isothiazolyl, pyridyl, pyridazinyl, pyrimidinyl, pyrazinyl, 1,3,5-triazynyl, phenyl, indazolyl, indolyl, indolizinyl, isoindolyl, benzo[b]furanyl, benzo[b]thiophenyl, (2,3-dihydro-1,4-benzodioxin-5-yl), benzimidazolyl, benzthiazolyl, purinyl, quinolynyl, isochinolyl, chinolyl, phtalazinyl, quinazolinyl, quinoxalinyl, 1,8-naphthyridinyl, pteridinyl, naphthyl or azulenyl, and preferably phenyl or (2,3-dihydro-1,4-benzodioxin-5-yl).

“Alkyl(C₁₋₃)” means methyl, ethyl, n-propyl or isopropyl.

“Alkyl(C₁₋₄)” means methyl, ethyl, h-propyl, isopropyl, n-butyl, 2-butyl, isobutyl or 2-methyl-n-propyl.

“Optionally substituted” means that a group may or may not be further substituted by at least one group chosen from alkyl, alkenyl, alkynyl, aryl, fluoro, chloro, bromo, hydroxyl, alkyloxy, alkenyloxy, aryloxy, acyloxy, amino, alkylamino, dialkylamino, arylamino, thio, alkylthio, arylthio, cyano, oxo, nitro, acyl, amido, alkylamido, dialkylamido, and carboxyl, or two optional substituents may together, with the carbon atoms to which they are attached, form a 5- or 6-membered aromatic or non-aromatic ring containing 0, 1 or 2 heteroatoms selected from nitrogen, oxygen and sulphur. Optional substituents may themselves bear additional optional substituents. Examples of optional substituents used in at least one embodiment of the present disclosure include C₁₋₃ alkyl, such as for example methyl, ethyl, and trifluoromethyl; fluoro; chloro; bromo; hydroxyl; C₁₋₃ alkyloxy, such as for example methoxy, ethoxy and trifluoromethoxy; and amino.

Prodrugs of the compounds mentioned above are within the scope of the present disclosure. Prodrugs are therapeutic agents which are inactive per se but are transformed into one or more active metabolites. Prodrugs are bioreversible derivatives of drug molecules used to overcome some barriers to the utility of the parent drug molecule. These barriers include, but are not limited to, solubility, permeability, stability, presystemic metabolism and targeting limitations (Medicinal Chemistry: Principles and Practice, 1994, ISBN 0-85186-494-5, Ed.: F. D. King, p. 215; J. Stella, “Prodrugs as therapeutics”, Expert Opin. Ther. Patents, 14(3), 277-280, 2004; P. Ettmayer et al., “Lessons learned from marketed and investigational prodrugs”, J. Med. Chem., 47, 2393-2404, 2004). Pro-drugs, i.e., compounds which when administered to humans by any known route, are metabolised to compounds having formula (1), belong to the invention. For example, in at least one embodiment, this relates to compounds with primary or secondary amino or hydroxy groups. Such compounds can be reacted with organic acids to yield compounds having formula (1) wherein an additional group is present which is easily removed after administration, for instance, but not limited to amidine, enamine, a Mannich base, a hydroxyl-methylene derivative, an O-(acyloxymethylene carbamate) derivative, carbamate, ester, amide or enaminone.

N-oxides of the compounds mentioned above are also within the scope of the present disclosure. Tertiary amines may or may not give rise to N-oxide metabolites. The extent to what N-oxidation takes place varies from trace amounts to a near quantitative conversion. N-oxides may be more active than their corresponding tertiary amines or less active. While N-oxides are easily reduced to their corresponding tertiary amines by chemical means, in the human body this happens to varying degrees. Some N-oxides undergo nearly quantitative reductive conversion to the corresponding tertiary amines; in other cases the conversion is a mere trace reaction or even completely absent. (M. H. Bickel: “The pharmacology and Biochemistry of N-oxides”, Pharmacological Reviews, 21(4), 325-355, 1969).

General Synthesis Details

The selection of the particular synthetic procedures depends on factors known to those skilled in the art such as the compatibility of functional groups with the reagents used, the possibility to use protecting groups, catalysts, activating and coupling reagents and the ultimate structural features present in the final compound being prepared.

Pharmaceutically Acceptable Salts

Pharmaceutically acceptable salts may be obtained using standard procedures well known in the art, for example by mixing a compound of the present disclosure with a suitable acid, for instance an inorganic acid such as hydrochloric acid, or with an organic acid.

Pharmaceutical Preparations

The compounds of the present disclosure can be brought into forms suitable for administration by means of usual processes using auxiliary substances such as liquid or solid carrier material. The pharmaceutical compositions of the present disclosure may be administered enterally, orally, parenterally (intramuscularly or intravenously), rectally or locally (topically). They can be administered in the form of solutions, powders, tablets, capsules (including microcapsules), ointments (creams or gel) or suppositories. Suitable excipients for such formulations are the pharmaceutically customary liquid or solid fillers and extenders, solvents, emulsifiers, lubricants, flavorings, colorings and/or buffer substances. Frequently used auxiliary substances which may be mentioned are magnesium carbonate, titanium dioxide, lactose, mannitol and other sugars or sugar alcohols, talc, lactoprotein, gelatin, starch, cellulose and its derivatives, animal and vegetable oils such as fish liver oil, sunflower, groundnut or sesame oil, polyethylene glycol and solvents such as, for example, sterile water and mono- or polyhydric alcohols such as glycerol.

Compounds of the present disclosure may be generally administered as pharmaceutical compositions which are important and novel embodiments of the present disclosure because of the presence of the compounds, more particularly specific compounds, disclosed herein. Types of pharmaceutical compositions that may be used include but are not limited to tablets, chewable tablets, capsules, solutions, parenteral solutions, suppositories, suspensions, and other types disclosed herein or apparent to a person skilled in the art from the specification and general knowledge in the art.

In one or more embodiments of the present disclosure, a pharmaceutical pack or kit is provided comprising at least one container filled with one or more of the ingredients of a pharmaceutical composition of the present disclosure. Associated with the at least container can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

Pharmacological Methods

Determination of MAO Activity

Neonatal rat striatal astroglial cells were used as a source for both MAO-A and MAO-B activity (Carlo et al., Brain Res 711, 175-183, 1996). Astroglial cells were cultured as described (Langeveld et al., Neurosci. Lett. 192, 13-16, 1995). Following one week in culture in 5% CO₂/95% air and at 37° C., cells were trypsinized and sonicated in ice cold 25 mM Tris-HCl buffer (pH 7.4) containing 1 mM EDTA. Thereafter, the obtained lysates were centrifuged for 5 min at 1 0,000 g and 4° C., and aliquots of the supernatant fractions were taken for determination of MAO activity using the Amplex Red MAO assay kit (Molecular Probes, Leiden, The Netherlands), which is based on the method described by Zhou and Panchuk-Voloshina (Anal. Biochem. 253, 169-174, 1997). Measurements were performed according to the manufacturers instructions. Briefly, prior to addition of the substrate, the samples were incubated for 30 min in a 96 well plate with drugs or solvent (total volume of 50 μl). Thereafter, 50 μl Amplex Red reagent was added containing 2 U/ml horseradisch peroxidase (HRP), p-tyramine HCl (2 mM, substrate for both MAO A and MAO B (Youdim and Finberg, Biochem. Pharmacol., 41, 155-162, 1991) and Amplex red (10 mM). Under these circumstances, via MAO-catalyzed oxidation of tyramine in a HRP-coupled reaction, Amplex red is converted to fluorescent resorufin. To measure MAO activity, the time-dependent increase in resorufin formation was determined at 2 min time intervals for 30 min at room temperature in a fluorescence microplate reader (BMG Labtechnologies GmbH, Germany), using excitation at 544 nm and emission at 595 nm. Within this time limit, the increase in fluorescence was found to be linear. For calculation of results, the data were corrected for background readings (i.e., in the absence of the MAO substrate tyramine), and expressed as the increase in arbitrary fluorescence units/min. Protein content was determined according to the method of Bradford et al., (Anal. Biochem. 72, 248-254, 1976), using BSA as a standard. Statistical comparisons between groups were made using a one-way analysis of variance (ANOVA) followed by a Newman-Keuls post-hoc test. P values <0.01 were considered significant.

MAO-B inhibitory activity of the compounds A1-D6 were measured at CEREP (Paris, France), according to the protocol described by J. L. Salach, Arch. Biochem. Biophys., 192, 128,1979.

Anethole dithiolethione and related dithiolethiones are active at doses in the range of 0.1-100 mg/kg after oral administration, and their selective inhibition of monoamine oxidase-B makes them particularly useful in the treatment of psychiatric and/or neurologic disorders caused by disturbances of the major monoaminergic systems or that can be treated via manipulation of those systems. These disorders include: mood disorders such as bipolar I disorders, bipolar II disorders and unipolar depressive disorders like minor depression, seasonal affective disorder, postnatal depression, dysthymia and major depression; anxiety disorders including panic disorder (with or without agoraphobia), social phobia, obsessive compulsive disorder (with or without co-morbid chronic tic or schizotypal disorder), posttraumatic stress disorder and generalized anxiety disorder; substance related disorders, including substance use disorders (like dependence and abuse) and substance induced disorders (like substance withdrawal); attention deficit and disruptive behavior disorders such as attention deficit hyperactivity disorder and narcolepsy; impulse control disorders like pathological gambling; eating disorders like anorexia nervosa and bulimia nervosa; tic disorders like Tourette's disorder; restless legs syndrome; disorders characterized by impairment of cognition and/or memory such as Alzheimers disease, Parkinson's disease and AIDS dementia and/or co-morbid psychiatric disorders and neurorehabilitation (post-traumatic brain lesions), other CNS disorders such as epilepsy, Down's syndrome, Huntington's disease, several forms of pain, including headache, atypical facial pain, pain disorder and chronic pain syndrome; amyotrophic lateral sclerosis and sexual dysfunction; disorders of the cerebral or peripheral vasculature, including essential, renovascular, pulmonary and ocular hypertension, thrombosis, myocardial infarction and cerebrovascular stroke; disorders of the non-vascular smooth muscle, including airway obstruction, asthma or another respiratory disorder and gastrointestinal motility disorders, hemorrhoids, sphincter and smooth muscle spasm in the gastrointestinal tract, and bladder dysfunction. Furthermore MAO inhibitors may counteract premature labor and relax the birth canal during delivery, are useful in relaxing the urinary tract for the passage of kidney stones, and maybe used to alleviate smooth muscle contraction and spasms.

Preferably, the compounds of the present disclosure are used for the treatment of mood disorders, bipolar I disorders, bipolar II disorders, unipolar depressive disorders, minor depression, seasonal affective disorder, postnatal depression, dysthymia, major depression, anxiety disorders, panic disorder, social phobia, obsessive compulsive disorder, posttraumatic stress disorder, generalized anxiety disorder, substance related disorders, substance use disorders, substance induced disorders, substance withdrawal, attention deficit and disruptive behavior disorders, attention deficit hyperactivity disorder, narcolepsy; impulse control disorders, pathological gambling, eating disorders, anorexia nervosa, bulimia nervosa, tic disorders, Tourette's disorder, restless legs syndrome, pain, headache, atypical facial pain, pain disorder and chronic pain syndrome, sexual dysfunction, airway obstruction, asthma, gastrointestinal motility disorders, hemorrhoids, sphincter and smooth muscle spasm in the gastrointestinal tract and bladder dysfunction.

Dose

The potency of the compounds of the invention as inhibitors of MAO-B was determined as described above. From the potency measured for a given compound of formula (1), one can estimate a theoretical lowest effective dose. At a concentration of the compound equal to twice the measured inhibition constant, 100% of the enzyme likely will be inhibited by the compound. Converting that concentration to mg of compound per kg of patient yields a theoretical lowest effective dose, assuming ideal bioavailability. Pharmacokinetic, pharmacodynamic, and other considerations may alter the dose actually administered to a higher or lower value. The dosage expediently administered is 0.001-1000 mg/kg, such as, for example, 0.1-100 mg/kg of patient's bodyweight.

Treatment

The term “treatment” as used herein refers to any treatment of a mammalian, including human conditions or diseases, and includes: (1) preventing the disease or condition from occurring in a subject which may be predisposed to the disease but has not yet been diagnosed as having it, (2) inhibiting the disease or condition, i.e., arresting its development, (3) relieving the disease or condition, i.e., causing regression of the condition, or (4) relieving the conditions caused by the disease, i.e., stopping the symptoms of the disease.

### EXAMPLES

### Example 1

### Materials and Methods

All reactions involving moisture sensitive compounds were carried out under a dry nitrogen atmosphere. Reactions were monitored by using thin-layer chromatography (TLC) on silica coated plastic sheets (Merck silica gel 60 F254) with the indicated eluent. The compounds were visualized by UV light (254 nm) or I₂. Flash chromatography refers to purification using the indicated eluent and Acros silica gel (0.030-0.075 mm). Nuclear magnetic resonance spectra (¹H NMR and ¹³C NMR, APT) were determined in the indicated solvent. Coupling constants J are given in Hz. Peakshapes in the NMR spectra are indicated with the symbols ‘q’ (quartet), ‘dq’ (double quartet), ‘t’ (triplet), ‘dt’ (double triplet), ‘d’ (doublet), ‘dd’ (double doublet), ‘s’ (singlet), ‘bs’ (broad singlet) and ‘m’ (multiplet).

### Example 2

### Synthesis of Specific Compounds

The specific compounds of which the synthesis is described below are intended to further illustrate the present disclosure in more detail, and therefore are not deemed to restrict the scope of the present disclosure in any way.

Structures of specific compounds of the present disclosure

| | |
|---|---|
| | (1) |
| 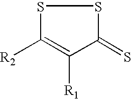 | |

| Comp | R₁ | R₂ |
|---|---|---|
| A1 | 4-hexyloxyphenyl | H |
| A2 | H | 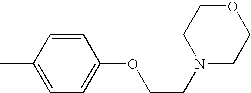 |
| A3 | H | 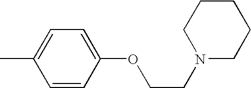 |
| B1 B2 | phenyl 4-methylphenyl | 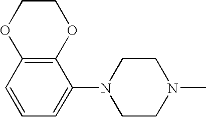 |
| B3 | 4-methylphenyl | 4-phenylpiperazinyl |
| C1 | H | —CH═CH-(4-diethylaminophenyl) |
| C2 | H | —CH═CH-(2-quinolinyl) |
| D1 | phenyl | —S(CH₂)₂CH(CH₃)NH-2-propynyl |
| D2 | phenyl | —S(CH₂)₃CH(CH₃)NH-2-propynyl |
| D3 | phenyl | —S(CH₂)₄CH(CH₃)NH-2-propynyl |
| D4 | phenyl | —S(CH₂)₄CH(CH₃)N(CH₃)-2-propynyl |
| D5 | phenyl | —S(CH₂)₃CH(CH₃)N(CH₃)-2-propynyl |
| D6 | phenyl | —S—CH₂-(4-methylphenyl) |

Other embodiments of the present disclosure will be apparent to those skilled in the art from consideration of the specification and practice of the present disclosure disclosed herein. It is thus intended that the specification and examples be considered as exemplary only, with a true scope and spirit of the invention being indicated by the claims.
Compound A1 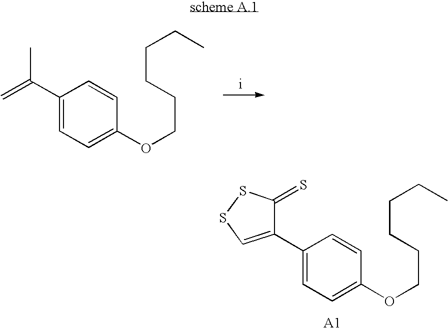
Step i Scheme A.1.

32 g (1 mol) of sulfur was added to 150 ml of DMF (N,N-dimethylformamide) and the resulting mixture heated to reflux until the sulfur nearly was dissolved. 43.6 g (200 mmol) of 2-(4-n-hexyloxyphenyl)-propene was added dropwise. After the addition was complete, stirring and heating was continued for the reaction followed by TLC (thin layer chromatography, eluent: toluene), after 4 hours the reaction was allowed to reach room temperature. Filtration and evaporation in vacuo of the reaction mixture yielded a residue which was subjected to column chromatography (SiO₂, eluent: toluene). The combined product containing fractions were concentrated in vacuo, the residue recrystallized from cyclohexane, yielding 5 g (8.1 %) of the desired compound A1. Melting point: 121° C.
Compound A2 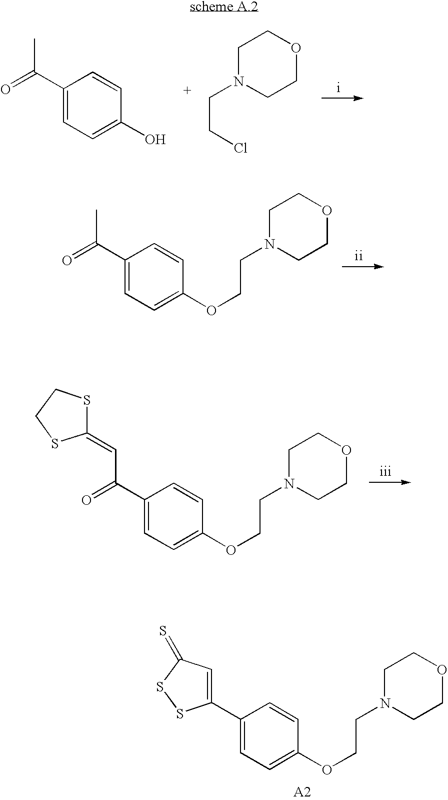
Step i Scheme A.2

To an absolute ethanolic solution containing 2 equivalents of sodium ethoxide (NaOEt) were added 1 equivalent of 4-hydroxyacetophenone together with 1 equivalent of N-(2-chloroethyl)morpholine. After addition was complete, the reaction mixture was allowed to reflux for 5 h, then heating was stopped and stirring continued for 12 h at room temperature. The solvent was removed in vacuo and the residue taken up in aqueous hydrogen chloride (ca. 2N), the latter solution was washed with diethylether. The aqueous layer was neutralized with sodium hydroxide solution (ca. 2N), after which extraction with diethylether was performed. The combined organic fractions were dried (Na₂SO₄). Removal of the drying agent by filtration and removal of the solvent in vacuo yielded the phenolic ether as an orange-yellow oil in 91% yield.

Step ii Scheme A.2 (according to Thuillier et al., Bull. Chim. Soc., (1959) 1398)

To a cold absolute amount of toluene containing 2 equivalents of sodium tert.-amylate (NaOC(CH₃)₂CH₂CH₃) were added 1 equivalent of the phenolic ether of step i, and 1 equivalent of carbondisulfide dissolved. When addition was complete, the reaction mixture was stirred for 6 h. Subsequently, 1 equivalent of 1,2-dibromoethane was added after-which stirring was continued for 12 h. The reaction mixture was washed with aqueous sodium hydroxide solution (ca. 2N) and with water until the pH reached 7. The organic fraction was dried on Na₂SO₄. Removal of the drying agent by filtration and removal of the solvent in vacuo yielded the pure 1,3-di-thiolane derivative as orange crystals in 70% yield.

Step iii Scheme A.2

The dithiolane derivative of step ii was treated with tetraphosphorusdecasulfide (P₄S₁₀) in refluxing xylene for 15 minutes. After cooling the suspension was washed with 1N sodium hydroxide (aq.) solution, after which chloroform was added, the resulting organic fraction was dried on Na₂SO₄. Removal of the drying agent by filtration and removal of the solvent in vacuo yielded a residue which was purified by column chromatography (SiO₂, eluent: diethylether/toluene 1/1), yielding the desired compound A2 as orange crystals in a yield of 4%. Melting point: 102° C. ¹H-NMR (CDCl₃, δ ppm): 2.59 (t, 4H), 2.83 (t, 2H), 3.73 (t, 4H), 4.17 (t, 2H), 6.98 (d, 2H), 7.60 (d, 2H), 7.36 (s, 1 H).

Compound A3 (red oil, ¹H-NMR (CDCl₃, δ ppm): 1.49 (q, 2H), 1.67 (t, 4H), 2.63 (t, 4H), 2.97 (t, 2H),4.24 (t, 2H), 7.00 (d, 2H), 7.38 (s, 1H), 7.60 (d, 2H)) was made analogously to the procedure described for compound A2.
Compound B1 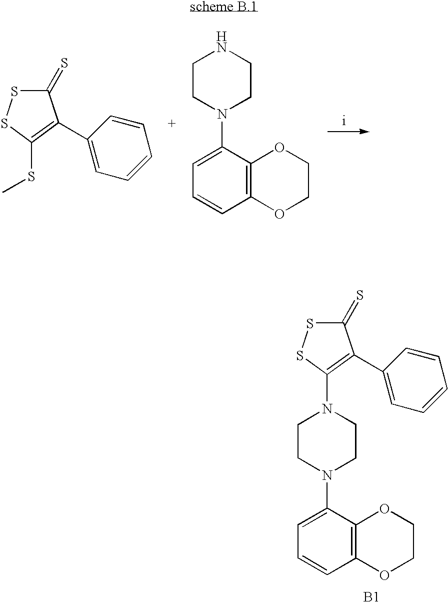
Step i Scheme B.1.

2 equivalents of the piperazine and 1 equivalent of 5-methylsulfanyl-4-phenyl-[1,2]-dithiole-3-thione (Grandin, A. et al., Bull. Soc. Chim. Fr., 11(1968)4555) were dissolved in absolute ethanol, after which the reaction mixture was brought to reflux temperature. After 7 days the solvent was removed in vacuo and the residue purified by chromatography (SiO₂, eluent: 2% ethanol in toluene v/v). The collected product containing fractions were concentrated in vacuo, the residue recrystallized from acetone, yielding orange crystals in 12% yield: compound B1. Melting point: 174° C. ¹H-NMR (CDCl₃, δ ppm): 2.97 (t, 4H), 3.44 (t, 4H), 4.23 (m, 4H), 6.42-6.76 (m, 3H), 7.34-7.49 (m, 5H).

Compound B2 (yellow crystals, melting point 108° C., ¹H-NMR (CDCl₃, δ ppm): 2.37 (s, 3H), 2.98 (t, 4H), 3.45 (t, 4H),4.23 (m, 4H), 6.43-6.76 (m, 3H), 7.22-7.28 (m, 4H)) was made analogously to the procedure described for compound B1.

Compound B3 (red crystals, melting point 148-150° C. with decomposition), was made analogously to the procedure described for compound B1.
Compound C1 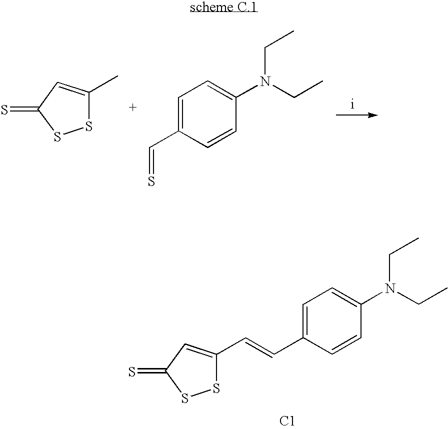
Step i Scheme C.1.

1 gram (6.8 mmol) of 5-methyl-[1,2]-dithiol-3-thion was dissolved in 50 ml of absolute ethanol. Then 2.5 gram (14.1 mmol) of 4-(diethylamino)-benzaldehyde and 1 ml of piperidine were added, after which the reaction mixture was heated on a water bath for 2 h. The reaction mixture was concentrated in vacuo and the residue placed in the freezer on which crystals formed. The crystals were isolated and recrystallized from isopropylalcohol yielding 1.5 gram (4.9 mmol, 35%) of the desired compound C1, melting point: 130° C. See also patent JP1319477.

Compound C2 (TLC (SiO₂, eluent: toluene), Rf=0.36, in presence of the staring materials), was made analogously to the procedure described for compound C1.
Compound D1 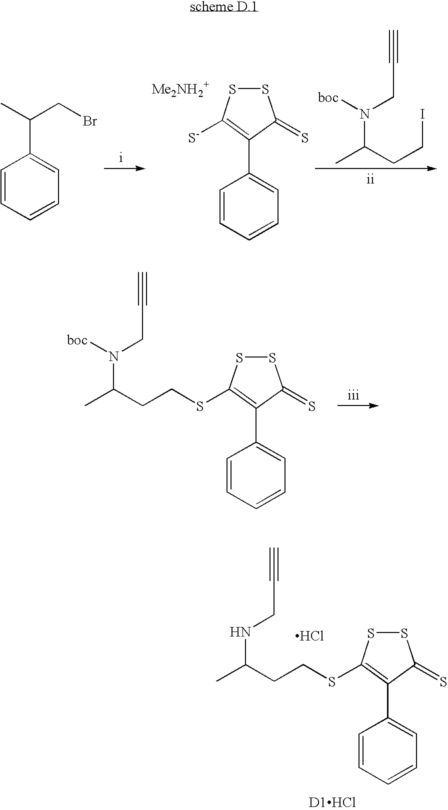
Step i Scheme D.1.

17.5 g (87.9 mmol) of 1-bromo-2-phenylpropane were dissolved in 300 ml of DMF after which 14.1 g (441 mmol) of sulfur were added. The reaction mixture was allowed to reflux for a night after which stirring was continued at room temperature for another night. The reaction mixture was concentrated in vacuo after which ca. 100 ml of toluene were added, crystals formed, the latter were collected and dried in vacuo. Yield: 13 g (45.3 mmol, 52%) of a yellow solid containing the dimethylammonium salt of the 4-phenyl-5-mercapto-[1,2]-dithiol-3-thion.

Step ii Scheme D.1.

3.5 g (10.4 mmol) of the iodide (for the synthesis of the iodide, vide infra) were dissolved in 40 ml of methanol after which 3 g (10.4 mmol) of the dimethylammonium salt (of step i) were added. The reaction mixture was stirred overnight, after which it was concentrated in vacuo, the residue being subjected to column chromatography (SiO₂, eluent: heptane/ethylacetate 6/1). Concentration of the product containing fractions yielded 700 mg (1.6 mmol, 15 %) of a red oil.

Step iii Scheme D.1.

700 mg (1.6 mmol) of the product of step ii was dissolved in a little dichloromethane after which a quantity of 7 N HCl (in isopropanol) was added, so that the end concentration was about 3N. The reaction mixture was stirred for a night after which it was concentrated in vacuo, yielding 265 mg of an orange solid containing D1.HCl, melting point: 243 ° C.

Compound D2 (melting point: 96-101° C., with decomposition), was made analogously to the procedure described for compound D1. The iodide which was used can be prepared to the synthesis of the iodide used for the preparation of D1 (see below).

Compound D3 (melting point: 82-87° C.), was made analogously to the procedure described for compound D1. The iodide which was used can be prepared to the synthesis of the iodide used for the preparation of D1.

Compound D4 (melting point: 65-70° C., with decomposition), was made analogously to the procedure described for compound D1. The iodide which was used can be prepared to the synthesis of the iodide used for the preparation of D1.

Compound D5 (melting point: 65-72° C.), was made analogously to the procedure described for compound D1. The iodide which was used can be prepared to the synthesis of the iodide used for the preparation of D1.

Compound D6 (melting point:134-135° C.) was made analogously to the procedure described for compound D1, starting with para bromomethyl toluene as the alkylating agent.

Synthesis of the iodide used in the preparation of compound D1 (scheme D.2). 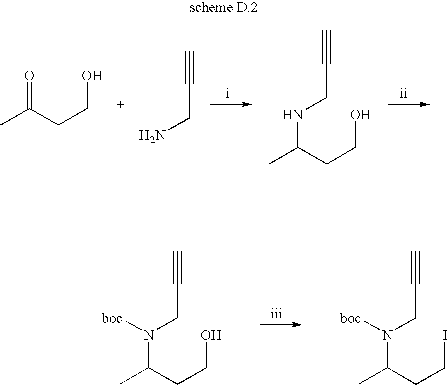
Step i Scheme D.2.

6.4 g (72.6 mmol) of 4-hydroxy-2-butanone was dissolved in 250 ml of 1,2-dichloroethane, after which 5.5 ml (80 mmol) of propargylamine was added. The reaction mixture was stirred for 10 minutes after which it was cooled to 0° C. 20 g (94 mmol) of NaBH(OAc)₃ were added portionwise to the reaction mixture, stirring was continued for 48 h, after which the mixture was poured into a saturated NaHCO₃ (aq.) solution. Extraction of the latter aqueous solution with DCM yielded 3 g of the desired product after concentration in vacuo. The aqueous layer was basified with NaOH solution (33%, aq.) and satutared with NaCl (solid), a second extraction with EtOAc was performed. The combined organic fractions were dried (Na₂SO₄) and after removal of the drying agent by filtration and removal of the solvent by concentration in vacuo, 7.6 g (82%) of the desired product (as an orange oil) was obtained. This was used in step ii without further purification.

Step ii Scheme D.2.

7.6 g (59.8 mmol) of the aminopropanol derivative (of step i) was dissolved in 200 ml of DCM after which 9.2 ml (˜65 mmol) of triethylamine and 14 g (65 mmol) of (Boc)₂O (Boc=tert.-butyloxycarbonyl) were added. The resulting mixture was stirred overnight after which it was concentrated in vacuo and the residue dissolved again in EtOAc. The organic fraction was washed with saturated NaHCO₃ (aq.) solution, water and brine, after which it was dried on Na₂SO₄. After removal of the drying agent by filtration and removal of the solvent by concentration in vacuo, 13.7 g (100%) of a brown oil, containing the N-Boc protected aminopropanol, was isolated.

Step iii Scheme D.2.

33 g (126. mmol) of triphenylphosphine were dissolved in 600 ml DCM, after which 19 g (280 mmol) of imidazole were added, the resulting mixture was brought to 0° C. A solution of 35.5 g (140 mmol) iodine in 300 ml of DCM (dichloromethane) was added dropwise to the reaction mixture, after which stirring was continued for 10 minutes. Subsequently, 8 g (35 mmol) of the N-Boc protected aminopropanol (from step ii) dissolved in 50 ml of DCM was added, stirring was continued at 0° C. for 20 minutes. Then the reaction mixture was allowed to reach room temperature and stirred for 16 h. The reaction mixture was filtered, the filtrate washed with brine and concentrated in vacuo. The residue was “filtered” over a short column of SiO₂ (eluent: heptane/EtOAc 6/1) and the eluate concentrated in vacuo, yielding 5.1 g (%) of the corresponding iodide as a light yellow oil. This iodide was used in the preparation of compound D1 (see scheme D.1).

The corresponding iodides needed for the preparation of compounds D2, D3, D4 and D5 can be prepared according to the conditions described in the synthesis of the iodide used in the preparation of D1 (scheme D.2). For compounds D4 and D5, the protection and deprotection steps (N-Boc) are not necessary because of the methyl group present on the nitrogen atom.

### Example 3

### Formulation of Compound A1

For oral (p.o.) administration: to the desired quantity (0.5-5 mg) of the solid compound A1 in a glass tube, some glass beads were added and the solid was milled by vortexing for 2 minutes. After addition of 1 ml of a solution of 1% methylcellulose in water and 2% (v/v) of Poloxamer 188 (Lutrol F68), the compound was suspended by vortexing for 10 minutes. The pH was adjusted to 7 with a few drops of aqueous NaOH (0.1N). Remaining particles in the suspension were further suspended by using an ultrasonic bath.

For intraperitoneal (i.p.) administration: to the desired quantity (0.5-15 mg) of the solid compound A1 in a glass tube, some glass beads were added and the solid was milled by vortexing for 2 minutes. After addition of 1 ml of a solution of 1 % methylcellulose and 5% mannitol in water, the compound was suspended by vortexing for 10 minutes. Finally the pH was adjusted to 7.

### Example 4

### Pharmacological Testresults

| | Σ MAO activity | | MAO-B activity | | | MAO-A activity | |
|---|---|---|---|---|---|---|---|
| | (% inhibition) | | (% inhibition) | | | (% inhibition) | |
| Conc. (μM) | L-dep | ADT | L-dep | ADT | D3T | Clor | ADT |
| 0.003 | 2 | — | 4 | — | — | — | — |
| 0.01 | 10 | — | 18 | — | — | — | — |
| 0.03 | 28 | — | 43 | 0 | — | — | — |
| 0.1 | 56 | 14 | 80 | 15 | — | 100 | — |
| 0.3 | 73 | 35 | 97 | 40 | — | — | — |
| 1 | 79 | 60 | 100 | 73 | 2 | — | — |
| 3 | — | 70 | — | 89 | 6 | — | 0 |
| 10 | — | 81 | — | 96 | 24 | — | 3 |
| 30 | — | 85 | — | 100 | 59 | — | 14 |
| 100 | — | — | — | — | 82 | — | — |
| 300 | — | — | — | — | 92 | — | — |
| 1,000 | — | — | — | — | 91 | — | — |

Effects of deprenyl (L-dep) and anethole dithiolethione (ADT) on total monoamine oxidase (Σ MAO) activity (columns 2 and 3); of L-dep, ADT and 3H-1,2-dithiole-3-thione (D3T) on monoamine oxidase-B (MAO-B) activity (columns 4, 5 and 6); of clorgyline (Clor) and ADT on monoamine oxidase A (MAO-A) activity (columns 7 and 8) in cellular extracts obtained from cultures of neonatal rat striatal astroglial cells. MAO activity and effects of the drugs thereon were determined as detailed above.

Data are expressed as percentages of respective controls, and are averages of two till five independent experiments performed in triplicate. Total MAO activity was determined in the presence of solvent (0.03% DMSO) only, MAO-B activity in the presence of 0.1 μM of the selective MAO-A inhibitor clorgyline, and MAO-A activity in the presence of 1 μM of the selective MAO-B inhibitor deprenyl.

Astroglial cells are known to express primarily MAO-B (Thorpe et al., J. Histochem. Cytochem., 35, 23-32, 1987). Using the non-selective substrate tyramine and the selective MAO-B inhibitor deprenyl (Youdim and Finberg, Biochem. Pharmacol., 41, 155-162, 1991), up to 80% of total astroglial MAO activity was found to consist of MAO-B. The remaining MAO activity (approximately 20%) in the presence of a maximally effective concentration of deprenyl, was inhibited completely by addition of the selective MAO-A inhibitor clorgyline. Deprenyl inhibited total astroglial MAO activity in a concentration-dependent manner with an apparent IC₅₀ of appr. 0.04 μM. Similarly, ADT concentration-dependently inhibited total MAO activity with an apparent IC₅₀ of approximately 0.5 μM, reaching a maximal effect (appr. 80% inhibition) at a concentration of 30 μM. Following selective and complete blockade of MAO-A activity by clorgyline, for both deprenyl and ADT identical concentration-effect relationships were observed as compared to those found for inhibition of total MAO activity. Under these conditions, i.e., in the presence of clorgyline, a similar, concentration-dependent blockade of MAO-B activity was observed for D3T, with an apparent IC₅₀ of approximately 20 μM and a maximally effective concentration of 300 μM. Upon selective and complete blockade of MAO-B activity with deprenyl, no statistically significant effect of ADT on MAO-A activity detected.

MAO-B inhibitory activity of the compounds A1-D6 AS measured at CEREP (Paris, France), according to the protocol described by J. L. Salach, Arch. Biochem. Biophys., 192, 128, 1979.

| Compound | MAO-B %-inhibition at 10⁻⁵ M |
|---|---|
| A1 | 100 |
| A2 | 81 |
| A3 | 83 |
| B1 | 65 |
| B2 | 74 |
| B3 | 54 |
| C1 | 52 |
| C2 | 56 |
| D1 | 25 |
| D2 | 100 |
| D3 | 95 |
| D4 | 81 |
| D5 | 75 |
| D6 | 71 |

1. A compound of formula (1), a tautomer, stereoisomer or N-oxide thereof, or a pharmacologically acceptable salt, hydrate or solvate of any of the foregoing: 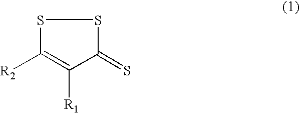
wherein:
R₁ is an optionally substituted phenyl; and R₂ is chosen from S—CH₂-(4-methyl-phenyl) and subgroups (i), (ii) and (iii): 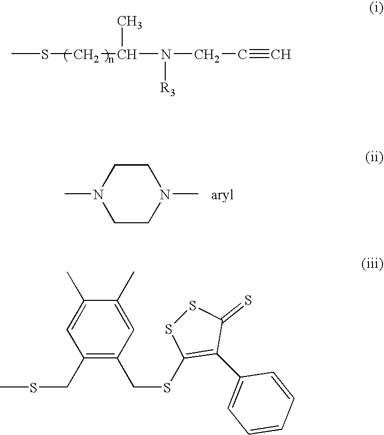
wherein n is chosen from 2, 3, 4 and 5; and R₃ is chosen from hydrogen and alkyl(C₁₋₃) groups; or
R₁ is 4-hexyloxyphenyl and R₂ is hydrogen; or
R₁ is substituted phenyl and R₂ is chosen from SH and subgroup (iv): 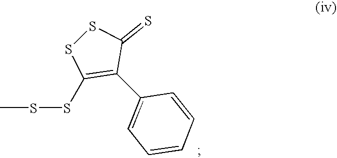
or
R₁ is hydrogen and R₂ is chosen from —CH═CH-4-(diethylaminophenyl), —CH═CH-(2-quinolyl) and subgroup (v): 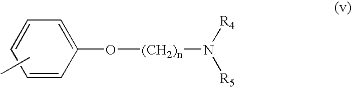
wherein n is chosen from 2, 3, 4 and 5; and R₄ and R₅ independently are chosen from alkyl(C₁₋₃) groups, or together with the nitrogen atom to which they are attached, form a saturated 5- or 6-membered ring, optionally containing a hetero-atom chosen from O, S, and an additional N; or
R₁ is chosen from alkyl(C₁₋₃) groups; and R₂ is 1-(2,3-dihydro-1,4-benzodioxin-5-yl)piperazin-4-yl; or
R₁ is cyano and R₂ is —NH—C(O)—NH-phenyl, wherein the phenyl group is optionally substituted; or
R₁ is —SO₂CH₃ and R₂ is chosen from amino groups.

2. The compound according to claim 1, a tautomer, stereoisomer or N-oxide thereof, or a pharmacologically acceptable salt, hydrate or solvate of any of the foregoing, wherein:
R₁ is 4-hexyloxyphenyl and R₂ is hydrogen; or 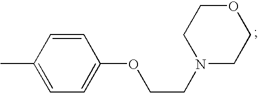
R₁ is hydrogen and R₂ is 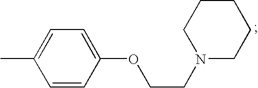
or
R₁ is hydrogen and R₂ is 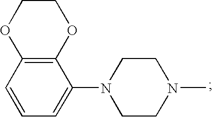
or
R₁ is phenyl and R₂ is 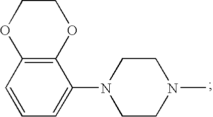
or
R₁ is 4-methylphenyl and R₂ is
or
R₁ is 4-methylphenyl and R₂ is 4-phenylpiperazinyl; or
R₁ is hydrogen and R₂ is —CH═CH-(4-diethylamino-phenyl); or
R₁ is hydrogen and R₂ is —CH═CH-(2-quinolinyl); or
R₁ is phenyl and R₂ is —S(CH₂)₂CH(CH₃)NH-2-propynyl; or
R₁ is phenyl and R₂ is —S(CH₂)₃CH(CH₃)NH-2-propynyl; or
R₁ is phenyl and R₂ is —S(CH₂)₄CH(CH₃)NH-2-propynyl; or
R₁ is phenyl and R₂ is —S(CH₂)₄CH(CH₃)N(CH₃)-2-propynyl; or
R₁ is phenyl and R₂ is —S(CH₂)₃CH(CH₃)N(CH₃)-2-propynyl; or
R₁ is phenyl and R₂ is —S—CH₂-(4-methylphenyl).

3. A pharmaceutical composition comprising:
a pharmacologically effective amount of at least one compound of formula (1), a tautomer, stereoisomer or N-oxide thereof, or a pharmacologically acceptable salt, hydrate or solvate of any of the foregoing: 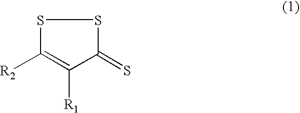
wherein:
R₁ and R₂ are the same or different, and are chosen from hydrogen, alkyl, alkenyl, alkynyl, aryl, fluoro, chloro, bromo, hydroxy, alkyloxy, alkenyloxy, aryloxy, acyloxy, amino, alkylamino, dialkylamino, arylamino, thio, alkylthio, arylthio, cyano, nitro, acyl, amido, alkylamido, and dialkylamido; or
R₁ and R₂, together with the carbon atoms to which they are attached, form a 5- or 6-membered aromatic or non-aromatic ring containing 0, 1 or 2 heteroatoms chosen from nitrogen, oxygen and sulphur;
R₁ and R₂ are optionally substituted with at least one substituent chosen from hydrogen, alkyl, alkenyl, alkynyl, aryl, fluoro, chloro, bromo, hydroxyl, alkyloxy, aminoalkyloxy, morpholin-4-yl-alkoxy, piperidin-1-yl-alkyloxy alkenyloxy, aryloxy, acyloxy, amino, alkylamino, dialkylamino, arylamino, thio, alkylthio, arylthio, cyano, oxo, nitro, acyl, amido, alkylamido and dialkylamido; and
at least one pharmaceutically acceptable carrier, at least one pharmaceutically acceptable auxiliary substance, or a combination thereof.

4. The pharmaceutical composition according to claim 3, wherein R₁ and R₂, together with the carbon atoms to which they are attached, form a 5- or 6-membered aromatic or non-aromatic ring containing 0, 1 or 2 heteroatoms chosen from nitrogen, oxygen and sulphur, said ring being chosen from furan, thiophene, pyrrole, oxazole, thiazole, imidazole, pyrazole, isoxazole, isothiazole, 1,2,3-oxadiazole, 1,2,3-triazole, 1,3,4-thiadiazole, pyridine, pyridazine, pyrimidine and pyrazine rings.

5. A method for preparing a pharmaceutical composition comprising: combining at least one compound of formula (1), a tautomer, stereoisomer or N-oxide thereof, or a pharmacologically acceptable salt, hydrate or solvate of any of the foregoing, with at least one pharmaceutically acceptable carrier, at least one pharmaceutically acceptable auxiliary substance, or a combination thereof: 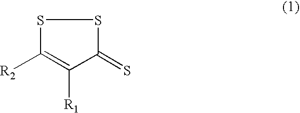
wherein:
R₁ and R₂ are the same or different, and are chosen from hydrogen, alkyl, alkenyl, alkynyl, aryl, fluoro, chloro, bromo, hydroxy, alkyloxy, alkenyloxy, aryloxy, acyloxy, amino, alkylamino, dialkylamino, arylamino, thio, alkylthio, arylthio, cyano, nitro, acyl, amido, alkylamido, and dialkylamido; or
R₁ and R₂, together with the carbon atoms to which they are attached, form a 5- or 6-membered aromatic or non-aromatic ring containing 0, 1 or 2 heteroatoms chosen from nitrogen, oxygen and sulphur;
R₁ and R₂ are optionally substituted with at least one substituent chosen from hydrogen, alkyl, alkenyl, alkynyl, aryl, fluoro, chloro, bromo, hydroxyl, alkyloxy, aminoalkyloxy, morpholin-4-yl-alkoxy, piperidin-1-yl-alkyloxy alkenyloxy, aryloxy, acyloxy, amino, alkylamino, dialkylamino, arylamino, thio, alkylthio, arylthio, cyano, oxo, nitro, acyl, amido, alkylamido and dialkylamido;
wherein the at least one compound of formula (1), a tautomer, stereoisomer or N-oxide thereof, or a pharmaceutically acceptable salt, hydrate or solvate of any of the foregoing is present in an amount effective for the treatment, amelioration or prevention of at least one disorder chosen from: mood disorders, bipolar I disorders, bipolar II disorders, unipolar depressive disorders, minor depression, seasonal affective disorder, postnatal depression, dysthymia, major depression, anxiety disorders, panic disorder, social phobia, obsessive compulsive disorder, posttraumatic stress disorder, generalized anxiety disorder, substance related disorders, substance use disorders, substance induced disorders, substance withdrawal, attention deficit and disruptive behavior disorders, attention deficit hyperactivity disorder, narcolepsy, impulse control disorders, pathological gambling, eating disorders, anorexia nervosa, bulimia nervosa, tic disorders, Tourette's disorder, restless leg syndrome, pain, headache, atypical facial pain, pain disorder and chronic pain syndrome, sexual dysfunction, airway obstruction, asthma, gastrointestinal motility disorders, hemorrhoids, sphincter and smooth muscle spasms in the gastrointestinal tract, and bladder dysfunction.

6. The method according to claim 5, wherein the at least one compound of formula (1) is chosen from 5-(p-methoxyphenyl)-3H-1,2-dithiole-3-thione, 3H-1,2-dithiole-3-thione and 4-methyl-5-(2-pyrazinyl)-3H-1,2-dithiole-3-thione.

7. The method according to claim 5, wherein the at least one compound of formula (1) is 5-(p-methoxyphenyl)-3H-1,2-dithiole-3-thione.

8. The method according to claim 5, wherein R₁ and R₂, together with the carbon atoms to which they are attached, form a 5- or 6-membered aromatic or non-aromatic ring containing 0, 1 or 2 heteroatoms chosen from nitrogen, oxygen and sulphur, said ring chosen from furan, thiophene, pyrrole, oxazole, thiazole, imidazole, pyrazole, isoxazole, isothiazole, 1,2,3-oxadiazole, 1,2,3-triazole, 1,3,4-thiadiazole, pyridine, pyridazine, pyrimidine and pyrazine rings.

9. A method for the treatment, amelioration or prevention of at least one disorder associated with the function of monoamine neurotransmission in a patient in need thereof, comprising:
administering a pharmacologically effective amount of at least one compound of formula (1), a tautomer, stereoisomer or N-oxide thereof, or a pharmaceutically acceptable salt, hydrate or solvate of any of the foregoing: 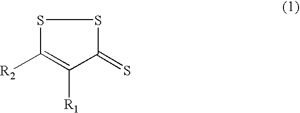
wherein:
R₁ and R₂ are the same or different, and are chosen from hydrogen, alkyl, alkenyl, alkynyl, aryl, fluoro, chloro, bromo, hydroxy, alkyloxy, alkenyloxy, aryloxy, acyloxy, amino, alkylamino, dialkylamino, arylamino, thio, alkylthio, arylthio, cyano, nitro, acyl, amido, alkylamido, and dialkylamido; or
R₁ and R₂, together with the carbon atoms to which they are attached, form a 5- or 6-membered aromatic or non-aromatic ring containing 0, 1 or 2 heteroatoms chosen from nitrogen, oxygen and sulphur;
R₁ and R₂ are optionally substituted with at least one substituent chosen from hydrogen, alkyl, alkenyl, alkynyl, aryl, fluoro, chloro, bromo, hydroxyl, alkyloxy, aminoalkyloxy, morpholin-4-yl-alkoxy, piperidin-1-yl-alkyloxy alkenyloxy, aryloxy, acyloxy, amino, alkylamino, dialkylamino, arylamino, thio, alkylthio, arylthio, cyano, oxo, nitro, acyl, amido, alkylamido and dialkylamido.

10. The method according to claim 9, wherein the at least one disorder is chosen from mood disorders, bipolar I disorders, bipolar II disorders, unipolar depressive disorders, minor depression, seasonal affective disorder, postnatal depression, dysthymia, major depression, anxiety disorders, panic disorder, social phobia, obsessive compulsive disorder, posttraumatic stress disorder, generalized anxiety disorder, substance related disorders, substance use disorders, substance induced disorders, substance withdrawal, attention deficit and disruptive behavior disorders, attention deficit hyperactivity disorder, narcolepsy, impulse control disorders, pathological gambling, eating disorders, anorexia nervosa, bulimia nervosa, tic disorders, Tourette's disorder, restless leg syndrome, pain, headache, atypical facial pain, pain disorder and chronic pain syndrome, sexual dysfunction, airway obstruction, asthma, gastrointestinal motility disorders, hemorrhoids, sphincter and smooth muscle spasms in the gastrointestinal tract, and bladder dysfunction.

11. The method according to claim 9, wherein the at least one compound of formula (1) is chosen from 5-(p-methoxyphenyl)-3H-1,2-dithiole-3-thione, 3H-1,2-dithiole-3-thione and 4-methyl-5-(2-pyrazinyl)-3H-1,2-dithiole-3-thione.

12. The method according to claim 9, wherein the at least one compound of formula (1) is 5-(p-methoxyphenyl)-3H-1,2-dithiole-3-thione.

13. The method according to claim 9, wherein R₁ and R₂, together with the carbon atoms to which they are attached, form a 5- or 6-membered aromatic or non-aromatic ring containing 0, 1 or 2 heteroatoms chosen from nitrogen, oxygen and sulphur, said ring being chosen from furan, thiophene, pyrrole, oxazole, thiazole, imidazole, pyrazole, isoxazole, isothiazole, 1,2,3-oxadiazole, 1,2,3,-triazole, 1,3,4-thiadiazole, pyridine, pyridazine, pyrimidine and pyrazine rings.
